(19) United States
(12) Patent Application Publication
(10) Pub. No.: US 2010/0113642 A1
(43) Pub. Date: May 6, 2010

(54) MOULDABLE, BIODEGRADABLE MATERIAL

(75) Inventors: Markku Leskelä, Espoo (FI); Timo Repo, Helsinki (FI); Petro Lahtinen, Helsinki (FI); Antti Pärssinen, Helsinki (FI); Jari Salo, Kerava (FI)

Correspondence Address: CHERNOFF, VILHAUER, MCCLUNG & STENZEL, LLP, 601 SW Second Avenue, Suite 1600, Portland, OR, 97204, US

(73) Assignee: INJECTOBONE FINLAND OY, Espoo (FI)

(21) Appl. No.: 12/593,224

(22) PCT Filed: Mar. 31, 2008
(86) PCT No.: PCT/FI08/50155

§ 371 Date: Dec. 8, 2009

(30) Foreign Application Priority Data
Mar. 30, 2007 (FI) 20075212

(51) Int. Cl. A61F 2/28; C08G 63/08; C08G 63/82

(52) U.S. Cl. 523/115; 528/354; 528/357

(57) ABSTRACT

A mouldable, biodegradable medical material, comprising an epsilon caprolactone homopolymer. The material is useful as an implant and in particular for filling irregularly shaped cavities in biological tissue in vivo. The epsilon caprolactone homopolymer can be produced by polymerizing epsilon caprolactone monomers in the presence of a titanium alkoxide catalyst.

The present invention concerns a biodegradable implant material according to the preamble of claim 1.

An implant of this kind generally comprises a biodegradable epsilon caprolactone polymer.

The present invention also relates to a method according to the preamble of claim 11 of producing an epsilon caprolactone polymer and to uses of the material.

Various implant materials are frequently employed in orthopaedia. There are a number of biocompatible implants available, e.g. for joint replacement, typically for total hip and knee replacement. There are also implants for replacement of bone parts and for treatment of bone defects, and for use in treatment of soft tissue, for fixing of other implants, tendons and ligaments to bones etc. Examples of such implants include rods and plates as well as fixing appliances, such as screws, spikes, sutures, threads and wires. The implant materials can roughly be divided in two groups depending on their biodegradability, viz. biostable (undegradable materials), such as titanium, surgical steel and bone cement, and biodegradable, which will degrade partially or totally in the biological environment of the human or animal body. The most common biodegradable implant materials include polylactide (PLA), polyglycolide (PGL) and polycaprolactone (PCL). Most of the commercially available implants manufactured from these biodegradable materials are nowadays used in preshaped form, for example as screws, plates, nets or threads (sutures, wires).

The mouldable, self-solidifying materials which are recommended for treatment of bone defects, replacement of removed segments of bone, for filling of cavities in bone matrices, now available are based on e.g. calcium triphosphate or hydroxyapatite. They are not hard or stiff enough to be used as a screw anchor or fixing aid. The most common self-reinforcing or autosolidifying material, bone cement, is primarily formed by poly(methyl methacrylate) (PMMA). There are at least two obvious problems relating to its use: first during manufacture toxic gases are released and second solidification of the material is an exothermal reaction which may generate so much heat that there can be local damage to the surrounding tissue. The PMMA is not a biodegradable material, as required in some applications. Being harder than bone, it can also have an abrasive effect on the bone in vivo. Generally, since PMMA is very tough compound, corrective actions after installation and hardening are difficult. It is also so hard that it is difficult to even drill after hardening.

Biodegradable materials for the above purposes are not in commercial use. One reason is that the mechanical properties and melt-processibility of known biodegradable materials are not always sufficient for demanding applications. Accordingly, there is a need for a fully and controllably biodegradable material that could be easily shaped into practically any desired form both for filling irregularly shaped cavities.

It is an aim of the present invention to eliminate at least a part of the problems associated with the known art and to provide a novel biodegradable implant as well as methods of producing materials suitable for implants and medical uses of such materials. In particular it is an aim of the invention to provide an implant material that can be heated and plasticized for application and which upon cooling solidifies into a mechanically durable solid implant which degrades in biological environment during a time period of about 1 month up to several years. It is also important that the surface layer of the applied material can be easily reshaped after initial hardening. This is sometimes necessary for providing space for surrounding tissues and other implants as well as in corrective actions.

The present invention is based on the finding that it is possible to produce biodegradable materials having excellent mechanical properties and good mouldability from homopolymers of epsilon caprolactone. The use of epsilon caprolactone monomers as comonomers in biodegradable materials containing significant amounts of lactide and/or glycolide monomers is known per se. However, there is no suggestion in the art that epsilon caprolactone homopolymers as such would be suitable for replacement implants of bones and bone defects and for treatment of defects in bone and soft tissue as a mouldable and hardening implant material.

In the present invention, implant materials are therefore provided which are based on epsilon caprolactone homopolymers. Such homopolymers typically have an inherent viscosity in the range from 0.4 to 1.9 dL/g. According to a particularly preferred embodiment, the homopolymer used in biodegradable, mouldable implant materials have an inherent viscosity between 0.7 and 1.0 dL/g.

These polymers can be produced by a method in which epsilon caprolactone monomers are contacted with a titanium alkoxide catalyst in liquid phase at elevated temperature. The implant materials are useful in various medical and veterinary applications. Particularly interesting is the use of the novel material for filling irregularly shaped cavities and as support materials for other implants in biological materials, such as bone.

More specifically, the mouldable, biodegradable implant according to the present invention is mainly characterized by what is stated in the characterizing part of claim 1.

The method according to the invention is characterized by what is stated in the characterizing part of claim 11.

The uses according to the invention are characterized by what is stated in the characterizing part of claim 15.

Considerable advantages are obtained by the invention. Thus, the novel materials are readily mouldable, as will be discussed in more detail, and can be used in a number of applications where the material needs to be shaped immediately before use. The materials also have such excellent mechanical properties that they can be employed as support materials for screws and prosthesis. Being readily produced in large quantities, the material is also suitable for use outside the human or animal body as a mouldable and hardening support material instead of conventional medical plaster.

The novel implant can be applied by injection or by spreading it out in the melt-phase, and it hardens upon cooling. The hardness and elasticity of the material can be adjusted by regulating the molecular weight of the polymer and the molecular weight distribution. Further, the biocompatibility of the implant, the porosity and solubility/dissolving in biological fluids and in biological environment can be modified by incorporating into the implant proper for example bioactive glass, soluble fibres, antibiotics and other biologically compatible and active materials.

Next the invention will be examined more closely with the aid of a detailed description and a number of working examples.

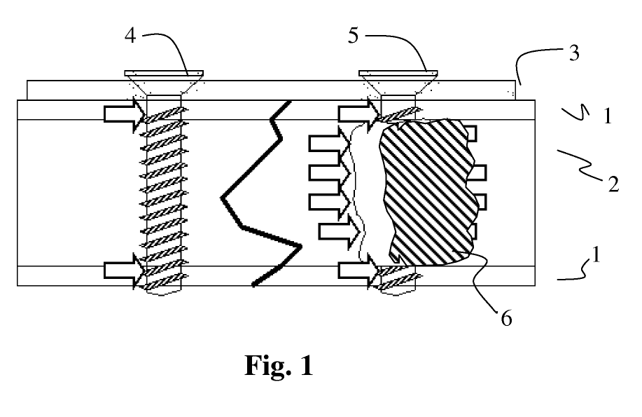

FIG. 1 is a schematic depiction of the use of the present materials for filling bone cavities, whereby a suitable matrix is provided for attachment of orthopaedic fastening means such as screws and pins;

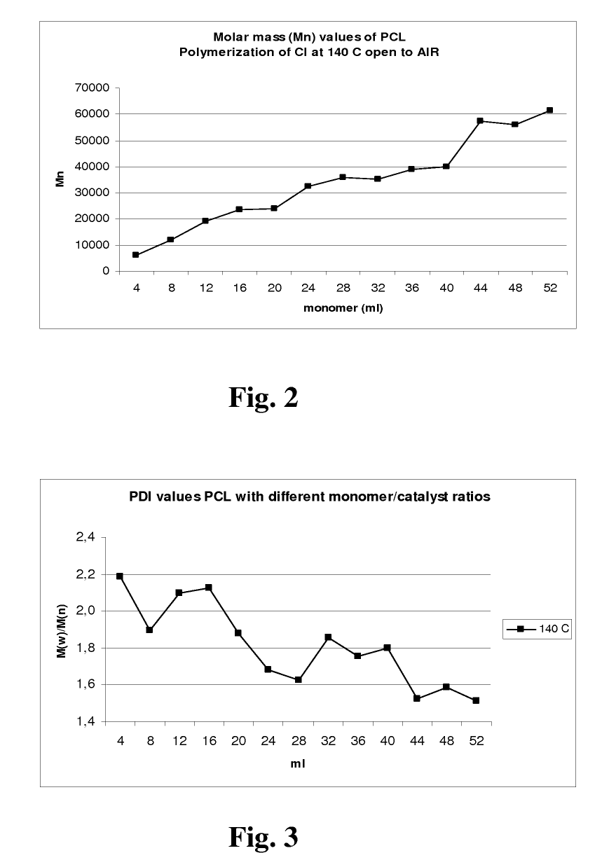

FIG. 2 shows the molecular weight (Mn) as a function of monomer/catalyst ratio;

FIG. 3 shows the polydispersity index (PDI) as a function of monomer/catalyst ratio;

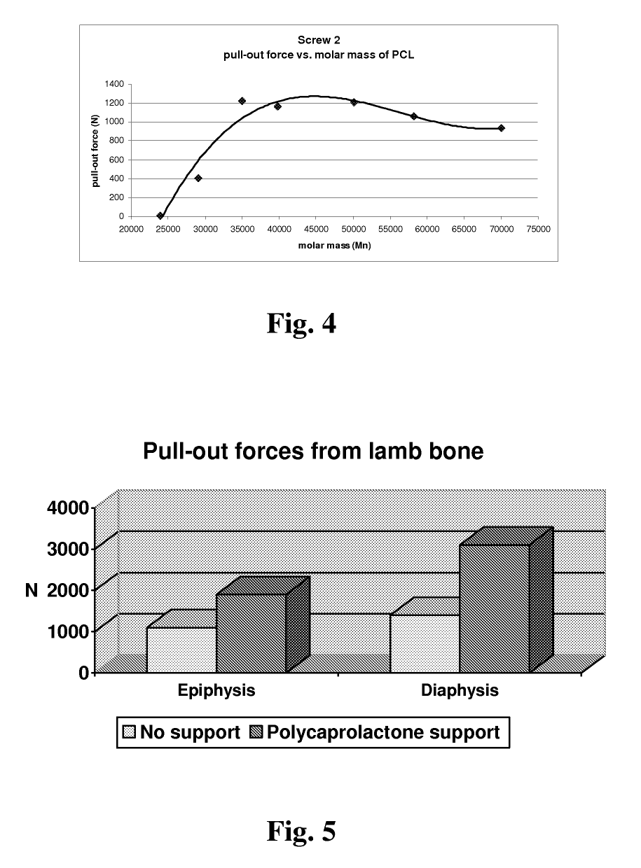

FIG. 4 shows the pull-out strength vs. molecular weight of an implant screw from polycaprolactone samples; and

FIG. 5 is a bar chart indicating the pull-out strengths of implant screw from lamb cortical bone.

The present mouldable, biodegradable medical material comprises an epsilon caprolactone homopolymer.

According to one embodiment, the epsilon caprolactone polymer is a homopolymer with a low inherent viscosity. The inherent viscosity of the homopolymer is at least about 0.4 dL/g, in particular at least 0.7 dL/g. Particularly interesting applications are with homopolymers having inherent viscosity values between about 0.8 and 1.0 dL/g.

According to another embodiment, the epsilon caprolactone polymer is a homopolymer with a reasonably broad molecular weight distribution. Therefore, the homopolymer preferably has a polymer dispersity index of at least about 1.2, in particular at least about 1.4. Particularly interesting applications are with homopolymers having a PDI of 1.5 or more, advantageously higher than 1.55, preferably about 1.6 to 5.

According to a third embodiment, the epsilon caprolactone polymer is a homopolymer which exhibits both low inherent viscosity and a reasonably broad molecular weight distribution, as indicated above.

It has been found that the epsilon caprolactone homopolymers will provide a combination of mouldability at relatively low temperatures and toughness and strength of the hardened material upon solidification which opens up the possibility to use it in particular as a biodegradable filling material.

The average molecular weight (Mₙ) of a suitable material is about 10,000 to 200,000 g/mol, in particular in the range of about 20,000 to 100,000 g/mol, preferably 20,000 to 80,000 g/mol, suitably about 25,000 to about 65,000 g/mol, advantageously about 35,000 to 60,000 g/mol. As regards the provision of a material having a preferred viscosity (cf. below) of about 1,000 to 2,000 Pas at 60° C., an average molecular weight of about 30,000 to 60,000 g/mol is particularly preferred.

The present material is typically a linear polymer which means that the degree of polymerization correspondence with the above molecular weight and amounts to about 50 to 2,000, in particular about 100 to 1,000, preferably about 200 to 500.

According to a further preferred embodiment, the material has an unsymmetrical molecular weight distribution. In practice, it is particularly preferred to have a polymer where the low molecular mass polymer portion is larger than the portion of the high molecular mass polymer.

According still to another preferred embodiment, the polycaprolactone has a broad molecular mass distribution, which in practice means that at least 5 mole-% of the polycaprolactone having a molecular weight of less than 25,000 g/mole and at least 5 mole-% of the polycaprolactone having a molecular weight of more than 60,000 g/mol. This embodiment of the invention can have a very broad molecular weight distribution (Mₙ interval 114 g/mol-200,000 g/mol). Typically, in the polycaprolactone (PCL), there is combined a low molecular weight PCL portion, for example having an average molecular weight of less than <25,000 g/mol, giving properties of good mouldability and the good mechanical durability of a PCL having a high molecular weight (for example PCL>60,000 g/mol).

The properties of the novel materials are interesting both with regard to their mechanical properties and their biodegradability. The material is typically manually mouldable at a temperature of 60° C. or less. According to one embodiment, an implant according to the invention is applied in the melt phase at a temperature of about 57 to 70° C. and it hardens at biological temperatures of about 35 to 43° C. to a mechanically durable solid implant. It can be applied manually or with an instrument, for example by injection.

According to another embodiment, an implant according to the invention is applied in the melt phase at a temperature of about 55 to 60° C.

For melt application, the (dynamic) viscosity at 60° C. should be below 10,000 Pas and preferably in below 5,000 Pas. A particularly preferred range is 1,000 to 2,000 Pas. This corresponds to an inherent viscosity of 0.7 to 1.0 dL/g.

The present invention also comprises a process for producing an epsilon caprolactone homopolymer having a polymer dispersion index of more than 1.5. This method comprises the steps of polymerizing epsilon caprolactone monomers in the presence of a titanium isopropoxide catalyst. It is preferred to continue the polymerization reaction so as to obtain to a polymer having an average molecular weight of at least 10,000 g/mol, preferably an average molecular weight of about 10,000 to 200,000 g/mol, as disclosed above.

In a conventional manner, the epsilon caprolactone homopolymer needs to be sterilized before use in a biological environment as an implant material. Sterilization can be carried out by thermal treatment, radiation or chemically, as known per se. Sterilization can be carried out immediately before use of the material, or the polymer material can be sealed into a suitable package and sterilized after packing.

The materials used in the present invention can be produced by conventional polymerization methods. Thus, the polymerization of the epsilon caprolactone monomers can be carried out in the melt phase or liquid phase as a conventional bulk polymerization by contacting the monomer at elevated temperature with a homogeneous catalyst. In order to produce a material having a broad molecular weight distribution, a catalyst comprising a titanium metal alkoxide is preferably used. Suitably the transition metal is titanium alkoxide having 1 to 6 carbon atoms. Preferred embodiments of such alkoxide groups are isopropoxide and n-butoxide. One particularly interesting catalyst is titanium isopropoxide. This catalyst can be used for polymerizing other cyclic hydroxyl acid monomers, also, e.g. for producing lactide homopolymers. Another example of a suitable catalyst is titanium n-butoxide.

The amount of the catalyst is about 0.001 to 2% calculated based on the volume of the epsilon caprolactone. By adjusting the monomer to catalyst ratio, it is possible to regulate the mechanical properties of the material and behaviour of the material in biological environment.

Results obtained in connection with the invention show that the preferred catalyst, titanium isopropoxide, primarily produces a homopolymer having a reasonably broad molecular weight distribution (PDI higher than 1.5). It is possible even further to broaden the distribution by incrementally adding monomer.

Biodegradability is an important feature since the implant is a non-living part inside the living body. As known, the implant should not be too rapidly degraded; typically a desirable degradation time ranges from several months up to years. Depending on the actual placement of the implant, a degradation time of 6 months to 36 months may be preferred. It has been found that such degradation times are achievable with the novel materials.

The polymerization temperature of the epsilon caprolactone monomers is higher than 100° C., preferably about 120 to 160° C. It is possible to operate the polymerization at reduced pressure or overpressure, although ambient pressure is preferred. With titanium-alcoxide catalysts, such as titanium-isopropoxide the polymerization can be carried out in an open reaction vessel without protective gas. In view of the non-demanding conditions of the polymerization, it is possible even to carry out polymerization in a surgical operating theatre/room.

Materials similar to the one obtainable by polymerization with a titanium isopropoxide catalyst can also be produced with known polymerization methods for example by controlling the feed of the epsilon caprolactone monomer during polymerization. Similar materials can be obtained also by suitable blending various commercially available PCL polymers.

The material discussed above can be used in medical implants for promoting regeneration of biological tissue. Such a material can be further blended with other components, such as polylactide and polyglycolide. When used with other polymers or as a block of a block copolymer, the portion of the present epsilon caprolactone homopolymer is still at least 20 mole-% of the total material composition, preferably the present homopolymer makes up at least 50 mole-% of the implant material, in particular at least 75 mole-%, and advantageously at least 85 mole-%.

However, it has been found that the strength and processability, in particularly ductility, toughness and strength in combination with mouldability, of the material are such that it can also be used as the sole matrix component of the implant.

Typical applications are surgical, medical, dental or veterinary treatment of the human or animal body.

The implant material can be processed into an orthopaedic appliance, optionally in the shape of a screw, a spike, pin, washer, thread or wire. The material can also be applied as a scaffold for bone repair in combination with biologically active materials, as will be explained below, and it can be used for production of elastic mats or tissues for cartilage, ligament or tendon repair.

The material can further be provided in the form of a solid block or slab of material which can be formed into pre-selected shape by melting the material which is applied in the molten state and allowed to solidify. A particularly interesting embodiment comprises a material which is applied to irregularly shape cavities as a filler and which can be used as a matrix for fastening of screws or pin or other orthopaedic fastening and repair means.

Turning now to the attached drawing, the conventional plate 3/screw 4—fixation in bone fractures typically is supported only by the hard cortex layers 1 of the long bones, as indicated by the two arrows on the left hand side. The limited force of screw fixation is a common problem in plate fixation, especially when used in osteoporotic bone 2.

By contrast, with the aid of the present invention, the attachment for of the screw 5 can be greatly increased. A material according to the present invention can be injected inside the bone 1 where it fills a cavity. Upon hardening, this material 6 is easily drilled to thread a screw in. Naturally, other fixation means can also be inserted into the filling mass/anchoring implant 6.

As discussed in the introduction, there are other anchoring methods of the above kind, but they employ non-resorbable materials which can be harmful in some situation inside the continuously remodelled bone tissue, or they are difficult to process and shape.

In addition to other structural components of biodegradable polymers, the present material can be mixed with other biocompatible materials, which are not necessarily biodegradable. The proportion of such biocompatible material is typically about 0.1 to 99, preferably about 0.1 to 50%, in particular about 1 to 30%, calculated from the total weight of the blend. The biocompatible materials can be biologically active material selected from the group of bone graft materials, such as bioactive glass and hydroxyapatite, drugs and hormones.

The biocompatible materials can also be inert materials which reinforce the implant.

The following non-limiting examples illustrate the invention.

### EXAMPLE 1

An epsilon caprolactone material was produced by heating 50 ml of ε-caprolactone at 140° C. under agitation. Titanium isopropoxide (catalyst) is added in an amount of 130 mikrolitre directly into the hot caprolactone liquid phase. The polymerization proceeds in 5 minutes up to the stage where the mixture starts gelling. Then the polymer is transferred into an oven where it is kept overnight at 100 until the degree of conversion is 99%. The molecular weight of the product was Mn=60,000-70,000 g/mol and PDI=1.7-2.0. The reaction was carried out in protective atmosphere.

### EXAMPLE 2

A predetermined amount, 3 ml, of ε-caprolactone was heated to 100° C. 130 microlitre of titanium isopropoxide was added and polymerization initiated. Further 47 ml of caprolactone was slowly added in such a way as to keep the material fluid the whole time (about during 6 minutes). When the material had gelled, it was transferred into an oven where it was kept at 100° C. until the conversion had risen to 99%. In this way, the PDI could be raised to about 2 while maintaining the molecular weight (Mn) at about 60,000 g/mol. This reaction was carried out without protective gas in an open reaction vessel.

### EXAMPLE 3

A predetermined amount, 20 ml, of ε-caprolactone was heated to 100° C. Titanium isopropoxide was added in an amount of 65 microlitre to initiate polymerization. After about 1 minute, another batch of 65 microlitre catalyst was added. When the viscosity suddenly increased, further 20 ml caprolactone was slowly added to keep the material in fluid state the whole time. After gelling the material was transferred into an oven where it was kept at 100° C. until conversion had risen to 99%. In this way, the PDI could be raised to higher than 2 while maintaining the molecular weight (Mₙ) to about 60,000 g/mol. This reaction is performed without protective gas in an open reaction vessel.

### EXAMPLES 4 TO 7

By repeating the methods of Examples 1 to 3, a number of caprolactone homopolymer compositions were produced having the properties indicated in Table 1.

TABLE 1

| | Protective | | | Viscosity | Strength at |
|---|---|---|---|---|---|
| Polymerization | gas | Mₙ | PDI | at 60° C. | 40° C. |
| Example 4 | yes | 60,000 g/mol | 1.7 | 10,000 Pas | 420 MPa |
| 120 μl catalyst | | | | | |
| 40 ml monomer | | | | | |
| Example 5 | yes | 50,000 g/mol | 1.8 | 2,700 Pas | 422 MPa |
| 150 μl catalyst | | | | | |
| 40 ml monomer | | | | | |
| Example 6 | yes | 40,000 g/mol | 1.7 | 860 Pas | 460 MPa |
| 200 μl catalyst | | | | | |
| 40 ml monomer | | | | | |
| Example 7 | no | 40,000 g/mol | 1.8 | 700 Pas | 400 MPa |
| 200 μl catalyst | | | | | |
| 40 ml monomer | | | | | |

In all the examples, the reaction temperature was 100 and the reaction time about 30 minutes. The catalyst was added in three portions of equal size with 2 minutes intervals (0 min, 2 min and 4 min).

### EXAMPLE 8

An epsilon caprolactone material was produced by heating (44 ml) of ε-caprolactone at 140° C. under agitation (open to air). During 10 minutes preheating the temperature of the solution increased to 123° C. and the amount of water in solution decreased to less than 10 ppm. Titanium isopropoxide (catalyst) is added in an amount of 140 μl directly into the hot caprolactone liquid phase. Polymerization was possible to initiate both with distilled and undistilled monomer. During the polymerization the temperature increases above 160° C. The polymerization proceeds in 5 minutes up to the stage where the agitation comes to a stop. After 10 minutes polymerization the degree of conversion of the monomer is over 95%. The molecular weight of the product was Mₙ=57,000 g/mol and PDI=1.52. Polymerization procedure can be carried out with different monomer/initiator ratio

### EXAMPLE 9

An epsilon caprolactone material was produced by heating (10 ml) of ε-caprolactone at 120° C. under agitation (open to air). Titanium n-butoxide (catalyst) is added in an amount of 200 μl directly into the hot caprolactone liquid phase. Further 33 ml caprolactone was slowly added to keep the material in fluid state the whole time. The polymerization proceeds in 5 minutes up to the stage where the agitation comes to a stop. After 10 minutes polymerization the degree of conversion of the monomer is over 96%. The molecular weight of the product was Mₙ=36,000 g/mol and PDI=1.68.

### EXAMPLE 10

An epsilon caprolactone material was produced by heating (52 ml) of ε-caprolactone at 120° C. under agitation (open to air). Titanium n-butoxide (catalyst) was added in an amount of 140 microlitre to initiate polymerization directly into the hot caprolactone liquid phase. After about 5 minute, another batch of 200 microlitre catalyst was added. The polymerization proceeds in 5 minutes up to the stage where the agitation comes to a stop. After 20 minutes polymerization the degree of conversion of the monomer is over 96%. The molecular weight of the product was Mₙ=34,000 g/mol and PDI=1.75.

### EXAMPLE 11

An epsilon caprolactone material was produced by heating (55 ml) of ε-caprolactone at 120° C. under agitation (open to air). Titanium-n-butoxide (catalyst) is added in an amount of 200 μl directly into the hot caprolactone liquid phase. During the polymerization the temperature increases to above 150° C. The polymerization proceeds in 10 minutes up to the stage where the agitation comes to a stop. The degree of conversion of the monomer is over 95%. The molecular weight of the product was Mₙ=51,500 g/mol and PDI=1.69.

The results of the polymerization are depicted in FIGS. 1 and 2, showing the development of molecular weight and PDI depending on monomer/catalyst ratio (see Example 8).

An analysis of commercial polymers showed that they have PDIs of 1.4 or less. Compared with such materials, the present materials provided better mouldability at 60° C. (viscosity below 1,000 Pas) while still exhibiting up to 25% greater hardness and tensile strength. (force needed for extending a stick of 3 times 2 mm was over 400 MPa).

### EXAMPLE 12

The applicability of the polycaprolactone as an implant screw anchor was studied. The pull-out strengths of implant screw from polycaprolactone samples were measured with Instron 4411. The screws were pulled out of cylinder shaped PCL-block (diameter 45 mm, thickness 20 mm) by constant speed of 10 mm/min. All the screws were inserted to a depth of 10 mm to the polycaprolactone cylinder.

The pull-out strengths are graphically presented in FIG. 4.

According to FIG. 4 the maximum pull-out strengths are observed from the polycaprolactone with molar mass of 35,000 g/mol to 55,000 g/mol. The inherent viscosities of these samples are between 0.69 dL/g and 0.91 dL/g.

### EXAMPLE 13

The applicability of the polycaprolactone as an implant screw anchor was further studied using a biological material (a lamb bone).

The pull-out strengths of implant screws supported with injected polycaprolactone from samples of a lamb bone were measured with Instron 4411. A hole for the implant screw was drilled (4.5 mm) to the bone and a self-threading implant screw was installed to the hole. The screws were pulled out from a lamb's cortical bone with a constant speed of 10 mm/min.

In the experiment where a polycaprolactone support was used, the polycaprolactone was injected into the hole prior to screw installation. Polycaprolactone with a molar mass of 50,000 g/mol and inherent viscosities of 0.9 dL/g was used as support material. The holes were situated in the epiphysis area and in the diaphysis area of the bone. The hole in the epiphysis area did not penetrate the back cortex of the bone. The diaphysis hole penetrated both cortexes of the bone.

The pull-out strengths of supported and unsupported implant screws are presented in FIG. 5. The left pillars represent pull-out strengths when implant screws were installed normally. The right hand columns represent pull-out strengths when polycaprolactone was injected to the hole made for the screw before the implant screw was installed.

As will appear from the figure, the pull-out strengths approximately doubled when a polycaprolactone support according to the present invention was used with the implant screw.

1. A mouldable, biodegradable medical material, comprising an epsilon caprolactone homopolymer and having an average molecular weight of 20,000 g/mol<Mₙ<100,000 g/mol.

2. The material according to claim 1, wherein the epsilon caprolactone homopolymer has a polymer dispersion index of more than 1.2.

3. The material according to claim 2, wherein the homopolymer has a polymer dispersion index of about 1.5 to 5.

4. The material according to claim 1, having an average molecular weight of 20,000 g/mol<Mₙ<80,000 g/mol, preferably 25,000 to 65,000 g/mol, in particular about 35,000 to about 60,000 g/mol.

5. The material according to claim 1, having an unsymmetrical molecular weight distribution.

6. The material according to claim 5, having a larger portion of a low molecular weight polymer than of a high molecular weight polymer.

7. The material according to claim 1, comprising polycaprolactone having a broad molecular weight distribution, at least 5 mole-% of the polycaprolactone having a molecular weight of less than 25,000 g/mole and at least 5 mole-% of the polycaprolactone having a molecular weight of more than 60,000 g/mole.

8. The material according to claim 1, being manually mouldable at a temperature of 70° C. or less, in particular 57 to 70° C.

9. The material according to claim 1, which can be moulded to allow for filling of irregularly shaped cavities.

10. The material according to claim 1, which is provided in sterilized form.

11. The material according to claim 1, exhibiting an inherent viscosity of 0.4 to 1.9 dL/g, in particular 0.7 to 1.0 dL/g.

12. A process for producing an epsilon caprolactone homopolymer according to claim 1, comprising polymerizing epsilon caprolactone monomers in the presence of a titanium alkoxide catalyst.

13. The process according to claim 12, wherein epsilon caprolactone monomers are polymerized at a temperature in excess of 100° C., preferably at 120 to 160° C., in a reaction vessel open to air.

14. The process according to claim 12, wherein epsilon caprolactone is polymerized by homogeneous catalysis, using 0.001 to 2% catalyst calculated from the volume of the epsilon caprolactone.

15. The process according to claim 12, wherein the catalyst is selected from titanium isopropoxide and titanium n-butoxide.

16. A medical implant for promoting regeneration of biological tissue, comprising a material according to claim 1.

17. The medical implant of claim 16, consisting essentially of a material that comprises an epsilon caprolactone homopolymer and having an average molecular weight of 20,000 g/mol<Mₙ<100,000 g/mol.

18. The medical implant of claim 16, for use in surgical, medical, dental or veterinary treatment of the human or animal body.

19. The medical implant of claim 16, comprising a solid block or slab of material which can be formed into preselected shape by melting the material which is applied in the molten state and allowed to solidify.

20. The medical implant of claim 19, comprising a solid piece of material which can be shaped to fill irregularly shaped cavities in human tissue, including bones and cartilage.

21. The medical implant of claim 16, comprising 0.1 to 99%, preferably about 1 to 50% biocompatible materials or mixtures thereof, blended with the biodegradable material, calculated from the total weight of the blend.

22. The medical implant of claim 21, wherein the biocompatible materials are biologically active material selected from the group of bone graft materials, such as bioactive glass and hydroxyapatite, drugs and hormones.

23. A medical plaster or external custom-made support for supporting bones and ligaments during tissue regeneration, or guiding to joint motion, comprising a material according to claim 1.

24. A support material for screws and prothesis in orthopaedic applications, comprising a material according to claim 1.

25. The support material according to claim 25, comprising an epsilon caprolactone homopolymer having an inherent viscosity of 0.4 to 1.9 dL/g, in particular about 0.7 to 1.0 dL/g.

26. A method of filling irregularly shaped cavities in biological tissue in vivo, comprising the steps of
heating a material according to claim 1 to make it mouldable,
introducing the mouldable material into the cavity, and
allowing the material to solidify inside the cavity.
